(19) United States
(12) Patent Application Publication
(10) Pub. No.: US 2001/0051155 A1
(43) Pub. Date: Dec. 13, 2001

(54) Methods and compositions for decreasing allergic reactions to surface allergens

(76) Inventors: Howard Sosin, Fairfeild, CT (US); Michael Caplan, Woodbridge, CT (US)

Correspondence Address: Choate, Hall & Stewart, Exchange Place, 53 State Street, Boston, MA, 02109, US

(21) Appl. No.: 09/897,864

(22) Filed: Jul. 2, 2001

Related U.S. Application Data
(63) Continuation of application No. 09/216,117, filed on Dec. 18, 1998.

(51) Int. Cl. A61K 39/395

(52) U.S. Cl. 424/130.1

(57) ABSTRACT

IgE binding epitopes on allergens which induce allergic symptoms following surface contact, such as those associated with latex rubber and cat allergies, among others, can be blocked. Molecules which bind to these epitopes can be identified and synthesized and then formulated to coat or blend with the allergenic surface to prevent patient IgE from gaining access to the allergenic epitopes. In one embodiment, the molecules are antibodies or antibody fragments which selectively bind to the epitopes that elicit the allergic response. In another embodiment, the masking reagents are peptides which mimic antibody fragments and bind to the relevant epitopes on the allergens. In a third embodiment, the cDNAs encoding Fab fragments which bind to the relevant antigens are isolated and the Fab proteins encoded by these cDNAs are prepared. These proteins can then be used in an assay to screen a combinatorial chemical library for compounds which bind to the relevant allergen and block the binding of the recombinant Fab fragments. These compounds are then used directly as masking reagents to block the IgE-binding epitopes on the relevant surface antigens. The masking compounds can be applied directly to or blended with the materials at the time or manufacture or later.

### BACKGROUND OF THE INVENTION

This invention is generally in the field of compositions to reduce allergic responses to surface allergens generally, such as latexes and other materials.

Allergic disease is a common health problem. Allergies exist to foods, molds, pollens, grasses, trees, insects, pets, fleas, ticks and other substances present in the environment. Some allergic reactions (especially those to foods and insects) can be so severe as to be life threatening. The majority of allergens discussed above elicit a reaction when ingested, inhaled, or injected. Allergens can also elicit a reaction based solely on contact with the skin. Animal fur is one common allergen. Another well known example is latex rubber which is used in many products such as medical supplies and personal protective equipment.

Latex rubber products are manufactured from a milky fluid derived from the rubber tree, Hevea brasiliensis and other processing chemicals. Proteins in latex rubber can cause a range of allergic reactions. Additionally, the proteins responsible for the allergic reactions can adhere to the powder placed in latex rubber gloves. This powder can be inhaled, causing exposure through the lungs. Two types of reactions can occur in persons sensitive to latex rubber: irritant contact dermatitis, and immediate systemic hypersensitivity. These reactions are mediated by IgE.

Proteins found in latex rubber that interact with antibodies have been characterized by two-dimensional electrophoresis. Protein fractions of 56, 45, 30, 20, 14, and less than 6.5 kd have been detected (Posch A. et al., (1997) J. Allergy Clin. Immunol. 99(3), 385-395). Acidic proteins in the 8-14 kd and 22-24 kd range that reacted with IgE antibodies were also identified (Posch A. et al., (1997) J. Allergy Clin. Immunol. 99(3), 385-395). The proteins, prohevein and hevein, from Hevea brasiliensis, are known to be major latex rubber allergens and to interact with IgE (Alenius, H., et al., Clin. Exp. Allergy 25(7), 659-665; Chen Z., et al., (1997) J. Allergy Clin. Immunol. 99(3), 402-409). The hevein lectin family of proteins has been shown to have homology with potato lectin and snake venom disintegrins (platelet aggregation inhibitors) (Kielisqewski, M. L., et al., (1994) Plant J. 5(6), 849-861).

The IgE binding domains have been shown mainly to be in the hevein fraction but epitopes also exist in the domain specific for prohevein (Chen Z., et al., (1997) J. Allergy Clin. Immunol. 99(3), 402-409). The main IgE-binding epitope of prohevein is thought to be in the N-terminal, 43 amino acid fragment (Alenius H., et al., (1996) J. Immunol. 156(4), 1618-1625).

Allergy is manifested by the release of histamines and other mediators of inflammation by mast cells which are triggered into action when IgE antibodies bound to receptors on the mast cell surface are cross linked by antigen. Other than avoidance, which is often problematic, and drug treatments (e.g. antihistamines, decongestants, and steroids) which only modify symptoms, the only currently medically accepted treatment for allergies is immunotherapy.

Immunotherapy involves the repeated injection of allergen extracts, to desensitize a patient to the allergen. Unfortunately, traditional immunotherapy is time consuming, usually involving years of treatment, and often fails to achieve its goal of desensitizing the patient to the allergen.

Initial trials with allergen-non-specific anti-IgE antibodies to deplete the patient of allergen-specific IgE antibodies have shown early promise (Boulet, et al. 1997; 155:1835-1840; Fahy, et al. American J Respir. Crit. Care Med. 1997; 155:1828-1834; Demoly P. and Bousquet J. American J Resp. Crit. Care Med. 1997; 155:1825-1827). On the other hand, trials utilizing immunogenic peptides (representing T cell epitopes) have been disappointing (Norman, et al. J. Aller. Clin. Immunol. 1997, 99:S127). Another form of allergen-specific immunotherapy which utilizes injection of plasmid DNA (Raz et al. Proc. Nat. Acad. Sci. USA 1994; 91:9519-9523; Hz et al. Int. Immunol. 1996; 8:1405-1411) remains unproven.

There remains a need for a safe and efficacious therapy for allergies, especially those where traditional immunotherapy is ill advised due to risk to the patient or lack of efficacy. There is also a need for alternatives to therapies, for example, by creating foods, materials or substances where the allergens that are most problematic are masked.

It is therefore an object of the present invention to provide a method for decreasing the allergenicity of allergens by producing a compound that will mask the relevant epitopes and thus prevent IgE from binding to them thereby mitigating the allergic response.

### SUMMARY OF THE INVENTION

IgE binding epitopes on allergens which induce allergic symptoms upon surface contact, such as those associated with latex rubber and cat allergens, among others, can be covered through interactions with blocking ligands referred to herein as “masking reagents”. Molecules which bind to these epitopes can be identified and synthesized as described below. These molecules are then formulated to neutralize the allergenic potential of surface proteins by preventing patient IgE from gaining access to the allergenic epitopes.

In one embodiment, the molecules are antibody fragments which selectively bind to the epitopes that elicit the allergic response. Standard techniques can be utilized to generate a combinatorial IgE library from mRNA isolated from the peripheral blood monocytes of patients allergic to a relevant surface allergen. This library can be screened by panning with the relevant antigen. Positive clones which produce recombinant Fab fragments specific for the relevant antigen are identified and the cDNA encoding the Fab fragment isolated. This cDNA can be used to drive the synthesis of large quantities of the recombinant Fab fragment, according to standard methods for the large scale preparation of recombinant proteins from transformed bacteria, yeast or insect cells or other high output systems for expression of recombination proteins. The recombinant Fab protein can be isolated and utilized directly as an agent to block the IgE binding epitopes of the relevant surface antigens.

Naturally occuring antibodies, either human or animal, or fragments thereof, can also be used which selectively bind to the epitopes that elicit the allergic response. Alternatively, hybridomas derived from patient peripheral blood monocytes which produce allergen-reactive IgE can be generated. If desired, standard methods can be used to prepare Fab fragments from these naturally occuring monoclonal antibodies, either through proteolysis or through cloning and recombinant expression. Once again, these Fabs can be used directly as blocking agents.

In another embodiment, the cDNAs isolated as described above are sequenced and the protein sequence corresponding to the 20-30 amino acids which participates in the antigen-binding hypervariable region determined. A peptide corresponding to this sequence will be synthesized chemically according to standard techniques or through standard methods for the large scale preparation of recombinant proteins. This peptide is used directly to block the IgE binding epitopes of the relevant surface antigens.

In a preferred embodiment, chemical compounds from a natural products chemical library or from a combinatorial synthetic chemical library that block the binding of patient IgE to specific epitopes will be identified or synthesized. To achieve this, the Fab fragments or monoclonal antibodies identified as described above are used in a screen for chemical compounds which block the binding of individual IgEs to each of the epitopes on the relevant allergen. The use of monoclonal antibodies or recombinant monoclonal Fabs rather than unfractionated IgEs in this assay permits the identification of compounds which block IgE binding to individual epitopes. These antibodies or antibody fragments are used in an assay to screen a combinatorial synthetic chemical library or natural products chemical library for compounds which bind to the relevant antigen and block the binding of the monoclonal antibodies or recombinant Fab fragments. These compounds are then used directly as agents to block the IgE-binding epitopes on the relevant surface antigens.

In all cases it is important to determine that the molecules used as blocking agents do not themselves cause an allergic response. This can be accomplished using standard techniques (i.e., skin tests, etc).

The masking compounds can be applied directly to or blended with the materials at the time of manufacture or later. Materials will typically be applied in a carrier that optimizes conditions for binding of the masking compounds to the epitopes. For example, in the case of antibody utilization, the antibodies or antibody fragments are suspended in a buffer at physiological pH or powder and then applied to the substrate for a period of time sufficient to bind the masking compounds. These materials may be a composition such as a latex formulation, or an animal, such as a dog or cat. In the case of latex gloves, the material is preferably applied in a powder or coating which is easily distributed throughout the glove or over the entire surface of the hand prior to insertion into the glove. The materials used could be combined with other substances to add fragrance or facilitate application or removal. In the case of cats, dogs, etc., enhancers which decrease dander or additives for making the hair glossy or smell better may be added. The compounds could also be put on surfaces that are likely to attract allergens, for example, walls, floors, draperies, carpets and furniture where animals or their fur and dander might settle. These can be applied using aerosol sprays, roll-ons, or other commonly available means.

### DETAILED DESCRIPTION OF THE INVENTION

Definitions

The following definitions are used herein.

An antigen is a molecule that elicits production of antibody (a humoral response) or an antigen-specific reaction from T cells (a cellular response).

An allergen is a subset of antigens which elicits IgE production in addition to other isotypes of antibodies.

An allergic reaction is one that is IgE mediated with clinical symptoms primarily involving one or more of the cutaneous (uticaria, angiodema, pruritus), respiratory (wheezing, coughing, laryngeal edema, rhinorrhea, watery/itching eyes), gastrointestinal (vomiting, abdominal pain, diarrhea), and cardiovascular (if a systemic reaction occurs) systems.

An epitope is a binding site comprised of an amino acid motif of between approximately six and fifteen amino acids, which can be bound by either an immunoglobulin or recognized by a T cell receptor when presented by an antigen presenting cell in conjunction with the major histocompatibility complex (MHC). A linear epitope is one where the amino acids are recognized in the context of a simple linear sequence. A conformational epitope is one where the amino acids are recognized in the context of a particular three dimensional structure.

A decreased allergic reaction is characterized by a decrease in clinical symptoms following treatment of symptoms associated with exposure to an allergen, which can involve respiratory, gastrointestinal, skin, eyes, ears and mucosal surfaces in general.

Many allergens are known that elicit allergic responses, which may range in severity from mildly irritating to life-threatening. Many of these allergens can be neutralized using the techniques described herein. Examples include the latex rubber proteins, animal hair and/or dander, especially domestic pets such as birds, dogs and cats, and livestock such as horses. Other examples include insect allergens, especially proteins from fleas, ticks, mites, ants, bees and cockroaches. Other exemplary allergens include molds, dust and pollen.

I. Masking Reagents

The preferred reagents are recombinant antibody fragments, synthetic peptides derived from the antibody hypervariable region, and synthetic molecules which mimic antibody binding to the epitopes, thereby blocking binding of the IgE. In all cases it is critical that the reagents do not themselves cause an allergic response and do not bind to IgE antibodies, nor crosslink Fcε receptors on mast cells, which would cause degranulation of the mast cells.

Recombinant IgE Fab Fragments

Standard techniques can be utilized to generate a combinatorial IgE library from patients allergic to a relevant surface allergen (Steinberger, S., et al. J. Biol. Chem. 271, 10967-10982 (1996)). This library can be screened by panning with the relevant allergen, as described by Steinberger, et al. Positive clones, which produce recombinant Fab fragments specific for the relevant antigen, are thereby identified and the cDNA encoding the Fab fragment can be isolated. This cDNA is used to drive the synthesis of large quantities of the recombinant Fab fragment, according to standard methods for the large scale preparation of recombinant proteins. The recombinant Fab protein is isolated and utilized directly as an agent to block the IgE binding epitopes of the relevant surface allergens.

A. Preparation and Screening of Libraries

There are two principal ways to obtain compounds which block IgE binding sites: combinatorial libraries and combinatorial chemistry.

### Identification of Compounds That Interact with IgE Binding Sites through Application of Combinatorial Phage Display Libraries Preparation and Screening of IgE Library

Steinberger et al. (Steinberger, P., Kraft D. and Valenta R. (1996) “Construction of a combinatorial IgE library from an allergic patient: Isolation and characterization of human IgE Fabs with specificity for the major Timothy Grass pollen antigen,” Phl p. 5 J. Biol. Chem. 271, 10967-10972) prepared a combinatorial IgE phage display library from mRNA isolated from the peripheral blood mononuclear cells of a grass allergic patient. Allergen-specific IgEs were selected by panning filamentous phage expressing IgE Fabs on their surfaces against allergen immobilized on the walls of 96 well microtiter plates. The cDNAs were than isolated from allergen-binding phage and transformed into E. coli for the production of large quantities of monoclonal, recombinant, allergen-specific IgE Fabs. This technique will be effective for both linear and conformational epitopes.

To determine whether the library screening has yielded a complete inventory of the allergen-specific IgEs present in patient serum, an immunocompetition assay can be performed. Pooled recombinant Fabs would be preincubated with immobilized allergen. After washing to remove unbound Fab, the immobilized allergen would then be incubated with patient serum. After washing to remove unbound serum proteins, an incubation with a reporter-coupled secondary antibody specific for IgE Fc domain would be performed. Detection of bound reporter would allow quantitation of the extent to which serum IgE was prevented from binding to allergen by recombinant Fab. The level of uncompeted serum IgE binding would be determined using allergen which had not been preincubated with Fab or had been incubated with nonsense Fab.

B. Production of Masking Compounds

### Antibody or Antibody Fragments

cDNA clones which produce recombinant Fab fragments specific for the relevant antigen will be used to drive the synthesis of large quantities of the recombinant Fab fragment, according to standard methods for the large scale preparation of recombinant proteins from a recombinant expression system. The recombinant Fab protein will be isolated and utilized directly as an agent to block the IgE binding epitopes of the relevant surface antigens.

Expression in a procaryotic or eucaryotic host including bacteria, yeast, and baculovirus-insect cell systems are typically used to produce large (mg) quantities of protein masking compound. Examples of methods for production in bacteria include Sporeno, et al., Cytokine 6(3), 255-264 (1994), and Packer, et al., Biotechnology (NY) 11(11), 1271-1277 (1993) (expression of “mini-antibodies” in E. coli), and of methods for production in mammalian cells include Werner, et al., J. Biotechnol. 22(1-2), 51-68 (1992).

Transgenic plants or animals can also be used to make recombinant protein masking compounds. Methods for engineering of plants and animals have been well known for a decade. For example, for plants see Day, (1996) Crit. Rev. Food Sci. & Nut. 36(S), 549-567, the teachings of which are incorporated herein. See also Fuchs and Astwood (1996) Food Tech. 83-88. Methods for making recombinant animals are also well established. See, for example, Colman, A “Production of therapeutic proteins in the milk of transgenic livestock” (1998) Biochem. Soc. Symp. 63, 141-147; Espanion and Niemann, (1996) DTW Dtxch Tierarztl Wochenschr 103(8-9), 320-328; and Colman, Am. J. Clin. Nutr. 63(4), 639S-6455S, the teachings of which are incorporated herein. All of these can serve as sources of the reagents.

### Hypervariable Peptide Sequences

The cDNAs encoding Fab fragments that bind to the relevant antigens are isolated as described above. These cDNAs can be sequenced and the protein sequence corresponding to the antigen-binding hypervariable regions determined. A peptide “antibody mimic” corresponding to this sequence will be synthesized chemically according to standard techniques. This peptide can be used directly to block the IgE binding epitopes of the relevant surface allergens. Generally speaking, the peptide will be between 10 and 15 amino acids long but could be as long as 30 amino acids. Alternatively, the peptide can be subjected to in vitro mutagenesis to identify alternatives which increase the binding affinity and/or stability.

### Synthetic Chemical Molecules

In some cases it may be preferable to utilize non-peptide compounds to block binding of IgE to the allergen by masking the IgE binding epitope. The cDNAs encoding Fab fragments that bind to the relevant allergens are isolated and the Fab proteins encoded by these cDNAs prepared as described above. These proteins can then be used in an assay to screen a combinatorial chemical library for compounds which bind to the relevant antigen and block the binding of the recombinant Fab fragments. These compounds will be used directly as agents to block the IgE-binding epitopes on the relevant surface antigens.

Antigen will be immobilized through adhesion to the wells of microtiter plates or through covalent linkage to a solid phase resin. Each well of the microtiter plate (or individual aliquots of the antigen-linked resin) will be incubated with a chemical compound. Each substance tested will be the product of a combinatorial chemical synthesis protocol or will be derived from a library of naturally occurring or synthetic chemical compounds. Unbound chemical compounds will be removed by rinsing the microtiter wells or resin aliquots in an appropriate buffer. Subsequently, a solution containing one of the antigen-specific Fab fragments, derived as described above, will be added to each well or mixed with each resin aliquot. After washing in buffer to remove unbound Fab fragments, a secondary antibody coupled to a detection reagent such as fluorescein or horseradish peroxidase and directed against human IgE Fab fragments will be added. Alternatively, standard molecular biologic techniques can be employed to couple a detectable signal entity such as green fluorescent protein to the Fab fragment itself. In this case, no secondary antibody incubation step will be required. Secondary antibody binding will be quantitated by measuring the detection signal. Those compounds which bind to and block an IgE binding epitope on the allergen will prevent the binding of the Fab fragment and hence of the detectable secondary antibody as well. Compounds of interest will thus be identified as those that reduce the detection signal.

It is critically important to demonstrate that any blocking compound achieves its blocking effect by binding to the epitope on the allergen rather than by binding to the IgE Fab fragment. While a compound which binds to the IgE Fab domain might be expected to block patient IgE binding to an allergen, it might also crosslink those patient IgE molecules, thus inducing the mast cell degranulation reaction which the application of the blocking compound was intended to prevent. Thus every compound identified through this assay is assayed for its ability to bind to the Fab fragment. Fab fragments are immobilized through covalent linkage to a solid resin. Fab resin is incubated with the compound of interest, after which unbound compound is removed by washing in an appropriate buffer. The Fab resin is then incubated with a radiolabelled version of the relevant allergen, and unbound allergen removed by washing in buffer. Antigen radiolabelling is achieved by biosynthetic labelling of bacteria with [³⁵S]-methionine (if the allergen is produced through recombinant means) or by enzyme-catalyzed radioiodination of native allergen with [¹²⁵I]. Allergen binding to the Fab-resin is quantitated by scintillation counting. If a compound of interest binds to the Fab fragment, it will prevent the binding of radiolabelled allergen. Thus, any compound which decreases the binding of radiolabelled allergen to the Fab resin must interact directly with the Fab fragment. Compounds which manifest this property will be discarded. This method could equally well be applied to immobilization of compound and testing for binding of the Fab fragments to the immobilized compound. Only those compounds which block Fab binding to allergen but do not bind directly to the Fab fragment are pursued further as potential therapeutically useful substances.

Identification of useful reagents can also be accomplished by using molecules that are selected from a complex mixture of random molecules in what has been referred to as “in vitro genetics” or combinatorial chemistry (Szostak, TIBS 19:89, 1992). In this approach a large pool of random and defined sequences is synthesized and then subjected to a selection and enrichment process, using screening techniques such as those described herein.

Chemical agents which are identified by the screening techniques outlined above can be prepared using standard synthetic chemistry.

Although described herein with reference to blocking agents-that is, compounds which bind to an epitope and prevent IgE from interacting, it will be possible in some cases to prevent IgE binding by using a blocking-agent that modifies the allergen rather than binds to it. For example, in the case of latex, it may be possible to add protein modifying denaturing reagents to the latex milk during the process of latex production. If these reagents alter the protein in such a way as to eliminate one or more epitopes without significantly altering the physical properties of the latex itself, they might serve as effective blocking reagents. Chemicals which reduce disulfide bonds and/or alkylate cysteines to prevent reformation of disulfide bonds, for example, might be expected to perturb epitopes, especially if these epitopes are conformational. Similarly, reagents which interact with amino groups (i.e. n-hydroxysuccinimide) or carboxylic groups (i.e. dansyl chloride) might affect IgE binding if the groups modified by these reagents contribute critically to an epitope.

II. Treatment of Materials

Masking compounds are applied to coat, or blended with, the material which elicits the allergic response. The masking compounds are applied to the intended materials to be treated and assayed to insure that the compound blocks binding sufficient to reduce patient allergic responses, preferably by at least 80%, more preferably by at least 90%, and most preferably by at least 98% of the IgE-binding mediated responses. The actual amount will be optimized for each allergen and reagent, using standard assays for IgE binding, such as ELISA and basophil assays.

Coating may be by absorption, adsorption, or binding through formation of covalent or hydrophobic bonds. Methods for chemically coupling proteins are well known and available from commercial suppliers such as Sigma Chemical Co., St. Louis, Mo. The masking compounds will typically be applied in an appropriate solvent, such as phosphate buffered saline for proteins, or an organic solvent for chemical compounds which are not soluble in aqueous solution.

For latex, blocking compound can be applied to a finished surface or blended into the latex milk during some stage of manufacture. For animals, blocking agents can be applied using aerosol sprays, rollons or other commonly available means, or added to some sort of shampoo or rinse with which the animal can be treated. Furthermore, the blocking compound could be applied to surfaces which come into contact with the pet or its fur, such as walls, floors, draperies, carpeting or furniture to prevent allergens adsorbed to these surfaces from eliciting allergic reactions. Thus, pet allergy symptoms can be treated, at least in part, without having to treat the pet. Additionally, formulating the blocking compound such that it can be applied to interior surfaces will be of particular benefit in treating insect allergies, especially to cockroaches, fleas, and dust mites, or allergies to molds and/or pollens. All available surfaces can be sprayed with an aerosol, or the air in the house passed through a treated filter, to help neutralize the allergenicity of the allergens deposited on carpeting, furniture, heating ducts, etc. The masking reagents can also be formulated in combination with insect control means, such as insecticides or growth inhibitors, which are then sprayed over the surfaces. In yet another embodiment, the masking reagents are applied to filters in enclosed places such as airplanes, to block allergens such as peanut proteins.

In an embodiment for application to animals, it is important that the carrier not be toxic to the animal, especially cats, since they groom themselves and will thereby ingest the applied coating. Typical carriers can include surfactants, adhesives, oils, scents, pigments, and other materials including propellants if applied in an aerosol. In an embodiment relevant to gloves, contraceptive devices, carpets, etc., it is important that the coating not be toxic and irritating to an individual in contact with the material.

Assays to assess an immunologic change after treatment with the masking compounds are known to those skilled in the art. Conventional assays include RAST (Sampson and Albergo, 1984), ELISAs (Burks, et al. 1986) immunoblotting (Burks, et al. 1988), and in vivo skin tests (Sampson and Albergo 1984). Objective clinical symptoms can be monitored before and after the administration of the treated material to determine any change in the clinical symptoms.

Modifications and variations of the methods and materials described herein will be obvious to those skilled in the art. Such modifications and variations are intended to come within the scope of the appended claims.

We claim:

1. A method for decreasing an allergic reaction to an allergen in a material by contacting the material with an effective amount of a reagent masking the epitopes in the allergen which bind to IgE, in an amount effective to reduce an allergic response in a person exposed to the material,
wherein the reagent is selected from the group consisting of antibodies or antibody fragments binding to the allergen, peptides corresponding to the hypervariable regions of antibodies which bind to the allergen, and synthetic chemical compounds binding to the allergen and
wherein the reagent does not bind to and crosslink IgE receptors on mast cells, and is not toxic or an irritant.

2. The method of claim 1 wherein the masking reagents are antibodies or antibody fragments reactive with IgE epitopes on protein allergens.

3. The method of claim 1 wherein the masking reagents are identified by screening of a recombinant library.

4. The method of claim 1 wherein the masking reagents are identified by screening of a combinatorial library.

5. The method of claim 1 wherein the masking reagents block latex rubber allergens.

6. The method of claim 1 wherein the masking reagents block animal allergens.

7. The method of claim 1 wherein the masking reagents are in a filter through which air or a vacuum is circulated.

8. The method of claim 1 comprising applying the masking reagent to interior surfaces.

9. A composition for decreasing an allergic reaction to a material comprising
a reagent masking the epitopes in the allergen which bind to IgE, in an amount effective to reduce an allergic response in a person exposed to the material,
wherein the reagent is selected from the group consisting of antibodies or antibody fragments binding to the allergen, peptides corresponding to the hypervariable regions of antibodies which bind to the allergen, and synthetic chemical compounds binding to the allergen and
wherein the reagent does not bind to and crosslink IgE receptors on mast cells, and is not toxic or an irritant, and
an appropriate carrier for contacting the material with the masking reagents.

10. The composition of claim 9 wherein the composition is suitable for application to animals.

11. The composition of claim 9 for coating of a latex material.

12. The composition of claim 9 wherein the masking reagents are immobilized in an air or vacuum filter.

13. The composition of claim 9 wherein the masking reagents are in a formulation for application to interior surfaces.

14. The composition of claim 9 wherein the masking reagents are chemical compounds modifying the IgE epitopes on latex proteins.

15. The composition of claim 14 in a powder or liquid form suitable for application to the surface of finished latex products directly contacting skin or other tissue surfaces.

16. The composition of claim 9 wherein the masking reagents bind to IgE epitopes on animal hair, feathers or dander.

17. The composition of claim 9 wherein the masking reagents bind to IgE epitopes in insect proteins, molds or pollens.
